(19) United States
(12) Patent Application Publication
(10) Pub. No.: US 2012/0070503 A1
(43) Pub. Date: Mar. 22, 2012

(54) Nanoparticle-Based Targeted Drug Delivery For In Vivo Bone Loss Mitigation

(75) Inventors: Hong DIXON, Helotes, TX (US); Qingwen NI, San Antonio, TX (US); Joseph A. McDONOUGH, Helotes, TX (US)

(73) Assignee: SOUTHWEST RESEARCH INSTITUTE, San Antonio, TX (US)

(21) Appl. No.: 12/886,334

(22) Filed: Sep. 20, 2010

(51) Int. Cl. A61K 9/50; A61P 19/08; A61K 31/5415; B82Y 5/00

(52) U.S. Cl. 424/497; 424/490; 514/226.2; 428/402; 977/773; 977/906

(57) ABSTRACT

The present invention is directed to nanoparticle-based targeted drug delivery system for treatment of bone-loss. An enantiomeric phenothiazine is formulated into an in-vivo nanoparticle delivery system which may contain bone-targeting functionality. The nanoparticle formulations and their associated influence on whole bone porosity may now also be evaluated utilizing nuclear magnetic resonance (NMR) and relaxation time profiles, and in particular, median T₂ relaxation times.

### FIELD OF THE INVENTION

The present invention is directed to nanoparticle-based targeted drug delivery system for treatment of bone-loss. An enantiomeric phenothiazine is formulated into an in-vivo nanoparticle delivery system which may contain bone-targeting functionality including other chemical characteristics to prolong blood circulation to achieve localized delivery of relatively high concentrations of antiresorptive compounds. The nanoparticle formulations and their associated influence on whole bone porosity may now also be evaluated utilizing nuclear magnetic resonance (NMR) and relaxation time profiles, and in particular, median T₂ relaxation times.

### BACKGROUND

Bone loss, osteoporosis, is recognized as a major health problem in the elderly, in individuals with genetic defects and in those who undergo prolonged periods of time in a weightless environment. For example, in the weightless environment, bone loss may occur at a level of about 2.0% per month due to decreased osteoblast activity without alteration in osteoclast activity. Significant bone loss may also occur in woman following estrogen removal. In the United States, osteoporosis is reportedly responsible for about 1.5 million fractures, 70,000 vertebral fracture, 250,000 wrist fractures and 300,000 fractures at other locations.

Osteopenia is a disease characterized by long term loss of bone tissue, particularly in the weight-supporting skeleton. Results of the joint Russian/US studies on the effect of microgravity on bone tissue from 4.5 to 14.5 month long missions have demonstrated that bone mineral density (BMD, g/cm²) and mineral content (BMC, g) are diminished in all areas of the astronaut skeleton. While osteopenia can affect the whole body, complications often occur predominantly at specific sites of the skeleton with great load bearing demands. The greatest BMD losses have been observed in the skeleton of the lower body, i.e., in pelvic bones (−11.99±1.22%) and in the femoral neck (−8.17±1.24%) while there was no apparent decay found in the skull region.

Similar results were found in the bed rest studies. In a −6 degrees head-down tilt 7-day bed rest model for microgravity, it was observed that there was a decreased bone formation rate in the iliac crest. To effectively countermeasure the bone loss, there is a standing need for a better therapeutic system that can deliver the required treatment within need-based and/or non-invasive type procedures.

### SUMMARY

A medicament comprising a phenothiazine having the structure:

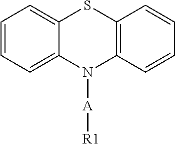

wherein A may be selected from the group consisting of linear or branched alkyls and/or linear or branched alkenyl groups having 1 to 5 carbon atoms; R1 may be a tertiary amine or thiol group having a structure including N—(R2)₃ or S—(R2) wherein R2 comprises the same or different entities selected from the group consisting of hydrogen, alkyl groups, alkenyl groups having 1 to 4 carbon atoms, cyclic alkene groups and heterocyclic alkylene groups comprising a heterocyclic element selected from the group consisting of nitrogen and sulfur. The medicament is provided in nanoparticle form having a largest linear dimension of 1-999 nanometers.

In another exemplary embodiment, the present disclosure relates to a method of preventing or inhibiting a disease or condition comprising administering to a patient or animal having a risk of having a disease or condition associated with bone loss, a therapeutically effective amount of a medicament comprising the phenothiazine described above.

In a still further exemplary embodiment of the present disclosure, a method for using nuclear magnetic resonance to characterize bone porosity is provided comprising placing a bone sample in an external magnetic field wherein the bone has a whole bone porosity comprising the porosity of the cortical, trabecular and marrow porosity for said bone. This may then be followed by providing an oscillating radio frequency electromagnetic field for exciting protons within the bone sample and providing a receiver to receive signals in the form of data from the excited protons. One may then measure the distribution of protons in the bone sample from the spectrum and process the data to characterize the whole bone porosity wherein the processing step includes determining the median T₂ relaxation times from the data.

In yet another exemplary embodiment, the present disclosure relates to a method of preventing or inhibiting a disease or condition comprising administering to a patient or animal having a risk of having a disease or condition associated with bone loss a therapeutically effective amount of a nanoparticle medicament including in-vivo bone targeting functionality comprising phenothiazines having the structure:

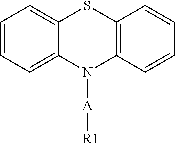

wherein A may be selected from the group consisting of linear or branched alkyls and/or linear or branched alkenyl groups having 1 to 5 carbon atoms; R1 may be a tertiary amine or thiol group having a structure including N—(R2)₃ or S—(R2) wherein R2 comprises the same or different entities selected from the group consisting of hydrogen, alkyl groups, alkenyl groups having 1 to 4 carbon atoms, cyclic alkene groups and heterocyclic alkylene groups comprising a heterocyclic element selected from the group consisting of nitrogen and sulfur; wherein the medicament is in nanoparticle form having a largest linear dimension of 1-999 nanometers and the nanoparticle form includes bone targeting functionality.

### BRIEF DESCRIPTION OF THE DRAWINGS

The detailed description below may be better understood with reference to the accompanying figures which are provide for illustrative purposes and are not to be considered as limiting any aspect of the invention.

FIG. 1A illustrates the size distribution of nanoparticles of (+)promethazine in PLGA.

FIG. 1B illustrates the controlled release of (+) promethazine at a pH of 7.4 in a phosphate buffered saline (PBS) at 37° C.

FIG. 2 illustrates the cumulative in vitro release of (+)promethazine-HCl from the nanoparticles at a pH 7.4 in a PBS at 37° C.

FIG. 3A illustrates the X-ray data of harvested rat cortical bone samples identified as “Bone 1”, “Bone 2” and “Bone 3” when using the indicated nanoparticles without a bone-targeting group. Bone 1 is HLS only, Bone 2 is HLS+IV (non-bone-targeting mixture of drug 1 and 2), Bone 3 is HLS+IV (non-bone-targeting mixture of drug 1 and 2+30 min loading).

FIG. 3B illustrates the X-ray data of harvested rat cortical bone samples identified as “Bone 1”, “Bone 2” and “Bone 3” when using the indicated nanoparticles with bone targeting. Bone 1 is HLS only, Bone 2 is HLS+IV (bone-targeting mixture of drug 4 and 5); Bone 3 is a normal control group.

FIG. 4A illustrates a NMR T2 relaxation time distribution spectra.

FIG. 4B illustrates the identification of the median T2 relaxation time of a NMR analysis of whole bone porosity.

### DETAILED DESCRIPTION

The present disclosure provides uses, medicaments and methods for reducing bone loss, e.g. treating periodontitis and osteoporosis, by administering a biologically or therapeutically effecting amount of an enantiomer of a chiral phenothiazine. The enantiomer is now preferably supplied in nanostructure form along with a biodegradable polymer that may include alendronate moieties (bisphosphonates) as one example of a bone targeting functionality. In addition, the nanostructures may comprise nanoparticles and the in-vivo formulations may include a polymeric component such as polyethylene glycol to prolong blood circulation and/or to provide localized delivery of relatively high concentrations of the chiral phenothiazine.

Reference to nanostructures herein may be understood as one or more solids having a largest linear dimension of 1-999 nm, including all values therein in 1.0 nm increments. Accordingly, the nanostructures may comprise nanoparticles having diameters of 1 nm, 2 nm, 3 nm, etc., up to 999 nm. The geometries contemplated therefore include round, oval, triangular, square, etc. As explained more fully herein, the nanoparticles may include encapsulated chiral phenothiazines for the indicated bone treatment protocols.

The chiral nature of the phenothiazine herein as used in nanoparticle form has now been confirmed for in actual vivo activity, and reference to such chirality is reference to the feature that the phenothiazine may exist as either the (+) or (−) enantiomer. However, although the (+) enantiomer now in nanoparticle form may have relatively higher efficacy for osteoclast inhibition in actual in vivo scenarios, the racemate and the (−) enantiomer may be utilized. Reference to (+) and (−) herein may be understood as optical rotation of plane polarized light as measured in water.

More specifically, the chiral phenothiazines now utilized may have the general structure:

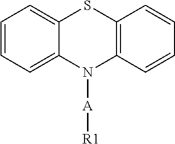

In the above, A may be selected from the group consisting of linear or branched alkyls and/or linear or branched alkenyl groups having 1 to 5 carbon atoms. R1 may be a tertiary amine or thiol group having a structure including N—(R2)₃ or S—(R2) wherein R2 comprises the same or different entities selected from the group consisting of hydrogen, alkyl groups, alkenyl groups, having 1 to 4 carbon atoms, cyclic alkene groups and heterocyclic alkylene groups comprising a heterocyclic element selected from the group consisting of nitrogen and sulfur.

Preferably, chiral phenothiazines may include promethazine, ethopropazine, propiomazine and trimeprazine. In one preferred embodiment, the chiral phenothiazine is the (+) enantiomer of promethazine of the structure:

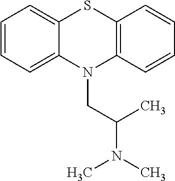

As discussed more fully below, the nanoparticles comprising the chiral phenothiazines disclosed herein may be formed and encapsulated with a polymeric component which polymeric component has hydrophilic and/or hydrophobic type character. Reference to hydrophilic may be understood as a polymer that has secondary attraction to water (e.g. the ability to H-bond with water) and reference to hydrophobic may be understood as a polymer that otherwise repels water (e.g. a polymer that is not capable of H-bonding with water).

For example, the polymer component may include poly(lactic-co-glycolic) acid, poly(lactic-b-PEG) and/or PLGA-alendronate polymers, which respectively may include the following general structures:

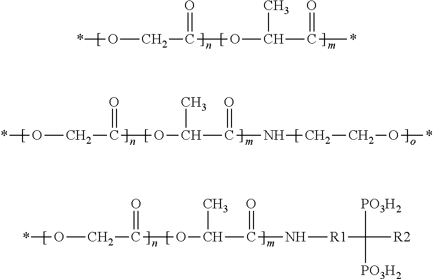

In the above formulas, the value of n, m and o may be any number between 1-1000 and R1 may comprise a linking functionality providing covalent attachment of the indicated bisphosphonate functionality, which linking functionality may specifically comprise an alkyl group such as (CH₂)ₓ where x has a value of 1-5. R2 may also comprise a hydroxyl group. Accordingly as now illustrated above, the bisphosphonate functionality, which provides for in-vivo bone targeting, may be attached via an ester type linkage and other linkages are contemplated herein such as amide linkages or urethane type linkages. Bone targeting functionality may be understood herein as any functionality having affinity for the bone, e.g., the extracellular inorganic matrix of the bone. Such affinity then allows for the bone-targeting functionality to deliver phenothiazines herein to the bone for interaction therewith.

The nanoencapsulated chiral phenothiazines may be preferably prepared by emulsion procedures. Specifically, emulsions may be prepared that can yield the nanoparticles herein, wherein the size, zeta potential, hydrophilicity and drug loading of the nanoparticles may be controlled by various parameters including the amount of emulsifier, drug and polymer and the intensity and duration of homogenization. As those skilled in the art can recognize, the single emulsion method may be employed for encapsulating hydrophobic drugs and a reverse emulsion or double emulsion method may be used for encapsulating hydrophilic drugs.

Because it is relatively difficult to investigate the precise mechanisms responsible for bone disuse, animal models were developed herein. More specifically, a reduced or zero lower limb weight-bearing disuse hind limb suspension (HLS) rat model was developed to conduct in-vivo investigations of bone loss and to confirm the in-vivo nanoparticle-based targeting drug delivery system disclosed herein.

More specifically, rat femurs were obtained and HLS preparations were initially performed for two tests with 4 weeks for each test. Details of the testing appear below. In general, the first test was to utilize the formulated drug herein, a (+)promethazine in PLGA without any targeting functionality, on 30 female rats:

5 for disuse only [5 rats, hind limb suspended only (as a control group)]

5 for disuse with drug [5 rats hind limb suspended and with IV injection of 0.1 mg/kg (+) promethazine]

5 for disuse+drug+30 min loading [5 rats hind limb suspended and IV injection of 0.1 mg/kg (+) promethazine and 30 min vibrations on the rat leg (30 HZ)]

5 for disuse+drug+60 min loading [5 rats hind limb suspended and IV injection of 0.1 mg/kg (+) promethazine and 60 min vibrations on the rat leg (30 HZ)]

5 for normal+drug [5 rats, no HLS, IV injection of 0.1 mg/kg (+) promethazine,]

5 for normal [5 rats, no HLS, as a control group]

The adaptive responses were evaluated following a four week period applied on 6 month old animals.

The second test herein was carried out using the same formulated drug but with targeting functionality (bisphosphonate) on 35 female rats (the dosage was again adjusted to 0.1 mg/kg): 5 for disuse only; 5 for disuse with drug (without targeting function), 5 for disuse with drug (with targeting function); 5 for disuse+drug+30 min loading; 5 for normal+drug (without targeting function); 5 for normal+drug (with targeting function), and 5 for normal.

After the first four weeks (drugs without targeting function) and the second four weeks (drugs including targeting function), the harvest cortical bone samples (right legs) were obtained from the rats. All the samples (right legs) were cleaned of soft tissues, and wrapped in calcium gauze and stored in separate containers filled with calcium buffered saline (CBS) and frozen at approximately −20° C. until testing.

### EXAMPLES

### (1) PLGA Nanoparticles with Encapsulated (+)Promethazine

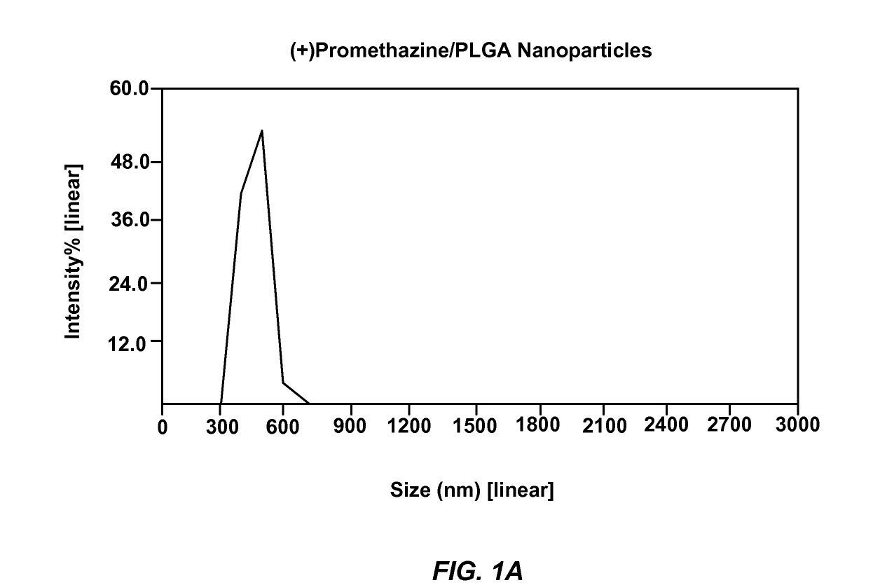

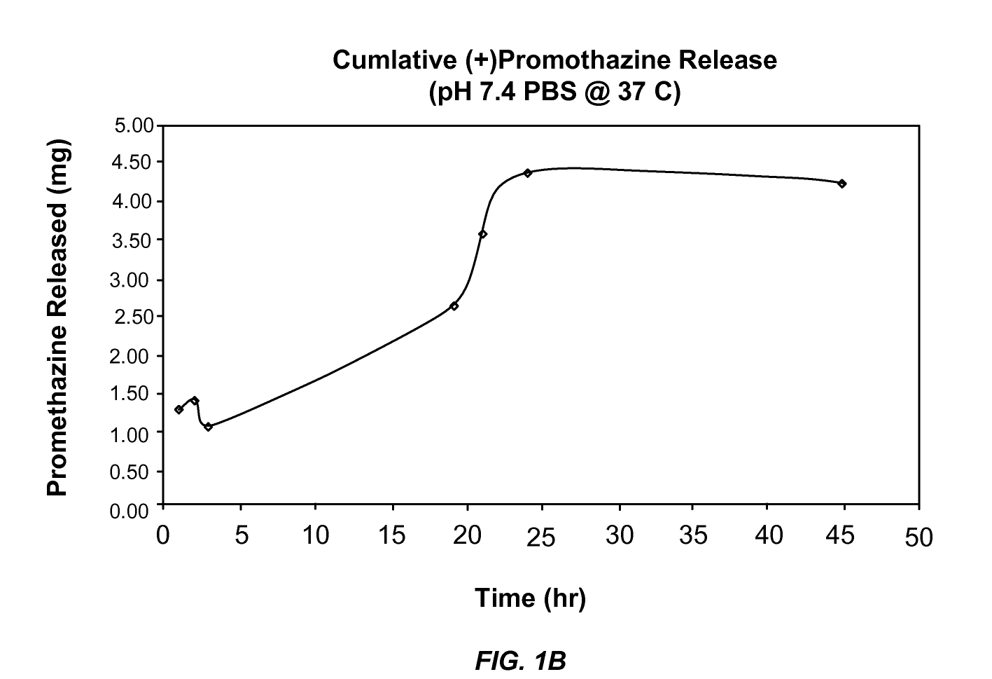

Nanoparticles of (+)promethazine in PLGA were initially prepared by the double emulsion method. The size distribution is illustrated in FIG. 1A. The positively charged nanoparticle samples demonstrated a controlled release of (+) promethazine for one day during in vitro testing. See FIG. 1B. The lyophilized nanoparticles can be re-suspended in pH 7.4 PBS. As may be seen, in vitro testing confirmed the controlled release of (+)promethazine.

### (2) PLGA and PLGA-b-PEG Copolymer Nanoparticles with Encapsulated (+)Promethazine

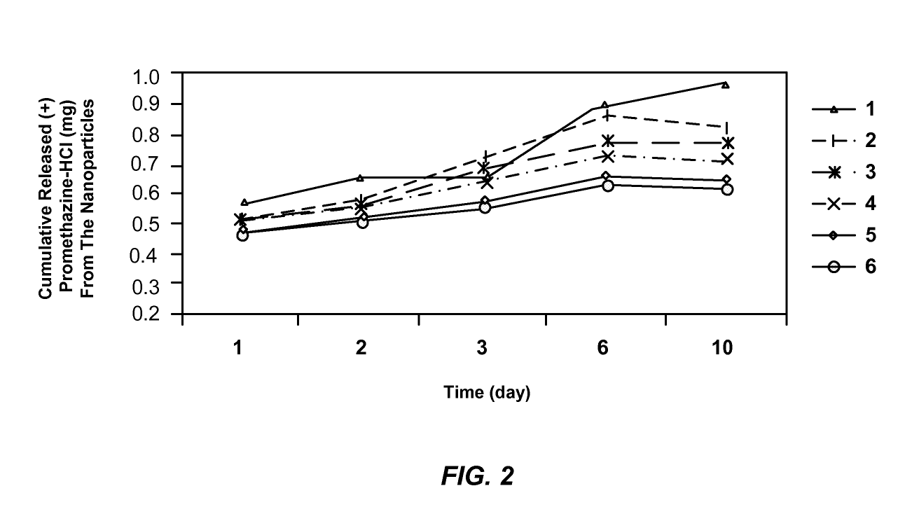

Nanoparticles of (+)promethazine in PLGA-PEG block copolymers were again prepared by a double emulsion method. The results are found in Table 1 and FIG. 2. As may be seen, in vitro testing again confirmed the controlled release of (+)promethazine.

TABLE 1
Nanoparticle Samples Used In Animal Studies

| | | (+) Promethazine | Zeta |
|---|---|---|---|
| Drug | | Payload (%, by | potential |
| Number | Composition | HPLC) | (mV) |
| 1 | 10 mg (+) promethazine•HCl | 29.1 | −38 |
| | 300 mg 5% PEG-PLGA | | |
| 2 | 10 mg (+) promethazine•HCl | 13.0 | −32 |
| | 300 mg 10% PEG-PLGA | | |
| 3 | 10 mg (+) promethazine•HCl | 14.7 | −39 |
| | 300 mg 15% PEG-PLGA | | |

### (3) PLGA-Alendronate and PLGA-b-Peg Copolymer Nanoparticles with Encapsulated (+)Promethazine

Nanoparticles of (+)promethazine/PLGA with bone-targeting moieties were prepared with alendronate conjugated PLGA polymers. The particle sizes of these samples were analyzed and they ranged between 50 and 200 nm. The zeta-potential and the payload of these samples were also analyzed by laser light scattering and HPLC respectively. See Table 2 and FIG. 2.

TABLE 2
Nanoparticle Samples Used In Animal Studies

| | | (+) Promethazine | Zeta |
|---|---|---|---|
| Drug | | Payload (%, by | potential |
| Number | Composition | HPLC) | (mV) |
| 4 | 10 mg (+) promethazine•HCl | 14.9 | −51 |
| | 200 mg 5% PEG-PLGA | | |
| | 100 mg PLGA-alendronate | | |
| 5 | 10 mg (+) promethazine•HCl | 19.0 | −38 |
| | 200 mg 10% PEG-PLGA | | |
| | 100 mg PLGA-alendronate | | |
| 6 | 10 mg (+) promethazine•HCl | 14.9 | −2 |
| | 200 mg 15% PEG-PLGA | | |
| | 100 mg PLGA-alendronate | | |

### (4) In-Vivo Testing Results

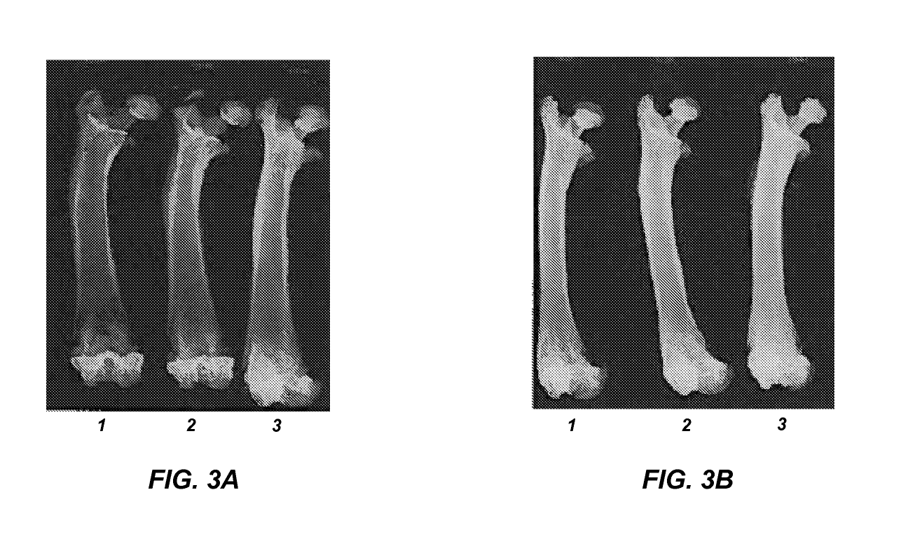

The six samples (details in Tables 1 and 2 and FIG. 2) were sent for in vivo testing. Age-matched rats were used in the HLS model. The dose used for the rats was 0.1 mg/kg every 48 hrs by intravenous treatment (IV). X-ray data of the harvested rat cortical bone samples can be found in FIGS. 3A and 3B.

More specifically, FIG. 3A shows the X-ray data of cortical bone samples for the IV treatment that employed nanoparticles without a bone-targeting group. Bone 1 as indicated was for HLS only; Bone 2 was for HLS+Drug 1/Drug 2; Bone 3 was for HLS+Drug 1/Drug 2+30 min loading. As can be seen (+) promethazine HCl was effective in preventing bone loss tested in the HLS model. Bone densities in bones 2 and 3 were higher than that of bone 1.

FIG. 3B shows the X-ray data of harvested rat cortical bone samples for the IV treatment that employed nanoparticles with bone targeting. Bone 1 as indicated was for HLS only; Bone 2 was for HLS+Drug 4/Drug 5; Bone 3 was for normal. As can be seen when the delivery of (+) promethazine HCl was targeted to the bone, its effectiveness in preventing bone loss was significant.

It may also now be appreciated that with respect to the use of the chiral phenothiazines herein, as a medicament for a condition relating to bone loss, such may be supplied as an implantable matrix or a transdermal delivery device. It may also be supplied in a controlled release oral carrier or in a pharmaceutically acceptable carrier.

NMR Testing

The present disclosure also relates to a nuclear magnetic resonance (NMR) testing protocol that may evaluate bone porosity. More specifically, it has now been found that median T2 relaxation times as measured by NMR are a useful parameter for whole bone porosity evaluation.

Reference to whole bone porosity evaluations may be understood herein as reference to the porosity of all of the following: (1) cortical bone; (2) trabecula; and (3) marrow bone. Reference to cortical bone may be understood as the cortex or outer shell of most bone that functions to support the body and protect organs and provide levers for movement, and which may store and release chemical elements, mainly calcium. Trabeculla bone may be understood as being relatively less dense, softer and weaker than cortical bone and that which typically occurs at the ends of relatively long bones proximal to joints and within the interior base of vertebrae. Trabelluar tends to be highly vascular and frequently contains red bone marrow where hematopoiesis may occur. Marrow bone may be understood as the flexible tissue found in the hollow interior of bones and which may include red marrow and yellow marrow.

A 0.5 to 40 MHz broadline NMR system was developed with an electromagnet having a 19 inch diameter with a 4 inch gap set up for a proton frequency of 27 MHz. A laboratory-built 1.0 inch diameter rf coil was also employed. ¹H spin-spin (T₂) relaxation profiles were obtained by using NMR CPMG {90° [−τ−180°−τ(echo)]ₙ−T_{R}} spin echo method with a 6.5 μs wide 90° pulse, τ of 500 μs, and T_{R} (sequences repetition rate) of 15 s. Each T₂ profile, one thousand echoes (one scan with n=1000) were acquired and forty scans were used. Thus, one scan will have repeated 1000 echoes in the window. The data was measured on fresh frozen human femurs after complete thawing in the room temperature (21±1° C.).

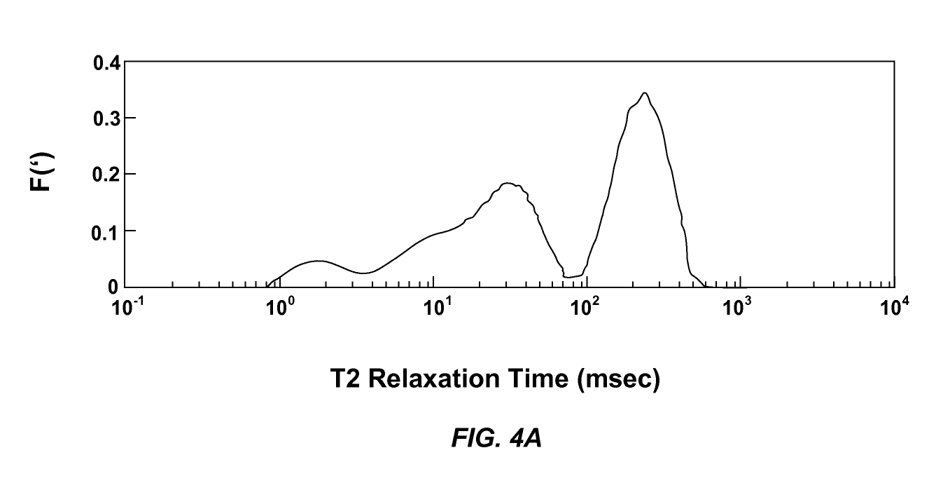

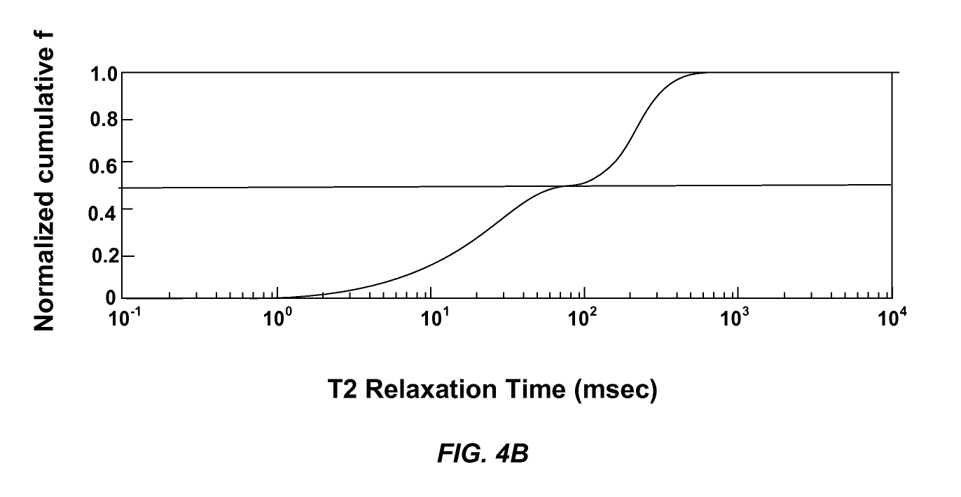

It was determined that the median T2 relaxation time as measured by NMR is a useful parameter for whole bone (cortical, trabecula, and marrow) porosity evaluations. In addition, NMR may now be used to effectively determine overall bone quality changes under various testing conditions for the animals (e.g. HLS, HLS+drug, HLS+drug+load, normal+drug, and normal only). The median T2 relaxation calculation is based on T2 relaxation distribution data. In T2 relaxation distribution spectra (FIG. 4A) the water intensity (amplitude in y axis) is plotted against T2 relaxation time (x-axis) which corresponds to different pore sizes and the cumulative water intensity amplitudes is normalized to 1. Therefore, the middle point 0.5 on y axis corresponds to the median relaxation time on x-axis. See FIG. 4B. This median relaxation time method can provide the whole relaxation mechanism without considering the bone size difference, i.e. different bone volumes for different bone. It is also a relatively sensitive method to analyze all pore size changes in an entire bone. NMR results for the bones from the animal study are summarized in Tables 3 and 4 below.

TABLE 3
Median Relaxation Times For Cortical Bone Samples
(Nanoparticles Without Bone-Targeting)

| | | | | Sample # | |
|---|---|---|---|---|---|
| | | Sample # | | (HLS + Drug1/ | |
| Sample | Median | (HLS + | Median | Drug2 + | Median |
| # | relaxation | Drug1/ | relaxation | 30 min | relaxation |
| (HLS) | (ms) | Drug2) | (ms) | loading) | (ms) |
| 126 | 69.11 | 131 | 50.54 | 136 | 44.81 |
| 127 | 49.65 | 132 | 52.80 | 137 | 42.93 |
| 128 | 75.66 | 133 | 52.88 | 138 | 63.78 |
| 129 | 67.77 | 134 | 57.28 | 139 | 72.04 |
| 130 | 51.24 | 135 | 45.12 | 140 | 39.19 |
| Average | 62.69 | | 51.72 | | 52.55 |

| Sample # | | | | | |
|---|---|---|---|---|---|
| (HLS + | Median | Sample # | | | |
| Drug1/Drug2 + | relax- | (Control + | Median | Sample # | Median |
| 60 min | ation | Drug1/ | relaxation | (Control | relaxation |
| loading) | (ms) | Drug2) | (ms) | only) | (ms) |
| 141 | 44.21 | 146 | 39.69 | 151 | 41.30 |
| 142 | 40.60 | 147 | 48.72 | 152 | 41.32 |
| 143 | 64.58 | 148 | 39.70 | 153 | 36.50 |
| 144 | 34.59 | 149 | 51.43 | 154 | 58.65 |
| 145 | 56.45 | 150 | 50.20 | 155 | 43.85 |
| Average | 48.09 | | 45.95 | | 44.32 |

TABLE 4
Median Relaxation Times For Cortical Bone Samples
(Nanoparticles With Bone-Targeting Groups)

| | | | | Sample | |
|---|---|---|---|---|---|
| Sample | Median | Sample # | Median | # | Median |
| # | relaxation | (HLS + Drug6 + | relaxation | (HLS + | relaxation |
| (HLS) | (ms) | 30 min loading) | (ms) | Drug3) | (ms) |
| 161 | 76.38 | 164 | 56.65 | 166 | 77.44 |
| 162 | 67.88 | 165 | 68.10 | 167 | 46.85 |
| 163 | 74.66 | 174 | 53.82 | 168 | 53.58 |
| 170 | 51.26 | 178 | 43.18 | 169 | 46.17 |
| 172 | 40.62 | 180 | 45.03 | 171 | 49.10 |
| Average | 62.16 | Average | 53.36 | Average | 54.63 |

| Sample # | | | | | |
|---|---|---|---|---|---|
| (HLS + | | Sample # | | | |
| Drug4/ | Median | (Control + | Median | Sample # | Median |
| Drug5 | relaxation | Drug4/Drug5 | relaxation | (Control + | relaxation |
| mixture) | (ms) | mixture) | (ms) | Drug3) | (ms) |
| 173 | 44.92 | 181 | 42.92 | 186 | 44.71 |
| 175 | 47.07 | 182 | 47.07 | 187 | 47.90 |
| 176 | 37.24 | 183 | 37.24 | 188 | 40.78 |
| 177 | 44.05 | 184 | 44.05 | 189 | 47.73 |
| 179 | 59.39 | 185 | 59.39 | 190 | 39.00 |
| Average | 46.53 | Average | 43.21 | Average | 44.02 |

| Sample # (Control only) | Median relaxation (ms) |
|---|---|
| 191 | 48.93 |
| 192 | 47.90 |
| 193 | 38.74 |
| 194 | 40.86 |
| 195 | 39.56 |
| Average | 43.20 |

The above confirms that a NMR method has now been developed to evaluate the effect of drug formulations on the degree of bone porosity. As explained more fully below, the NMR results above were observed to correlate well with the X-ray data. The use of average median relaxation time is now clearly shown to be valuable in assessing bone porosity. See FIGS. 3A and 3B and Table 3.

The first animal study demonstrated the efficacy of nanoencapsulated (+)promethazine. HCl in reducing bone loss under microgravity conditions in rats by the HLS protocol. The average median relaxation is reduced to 51.72 ms with the drug treatment from 62.69 ms without drug treatment. The added loading showed further improvement at 60 min (48.09 ms) but not at 30 min (52.55 ms). Applying the drug formulation to non-HLS treated animals (45.95 ms) showed no effect compared to the control animals (44.32 ms).

The second animal study demonstrated better efficacy of the drug formulation with targeting functional groups. The average median relaxation is reduced to 46.53 ms with the drug treatment from 62.16 ms without drug treatment. Again applying this drug formulation to non-HLS treated animals (43.21 ms) showed no effect compared either to the control animals (43.20 ms) or to the animals treated with a formulation without targeting functions (44.02 ms).

The two animal studies demonstrated reproducible results can be obtained with the rat HLS model. In addition, the controlled release of (+) promethazine.HCl from the developed nanoparticle formulations showed antiresorptive efficacy in the animals under simulated microgravity conditions and the efficacy can be further improved with bone-targeting functional groups on the nanoparticles or with 60 min loading.

What is claimed is:

1. A medicament comprising phenothiazines having the structure: 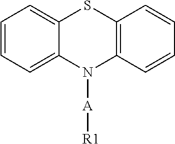
wherein A may be selected from the group consisting of linear or branched alkyls and/or linear or branched alkenyl groups having 1 to 5 carbon atoms;
R1 may be a tertiary amine or thiol group having a structure including N—(R2)₃ or S—(R2) wherein R2 comprises the same or different entities selected from the group consisting of hydrogen, alkyl groups, alkenyl groups having 1 to 4 carbon atoms, cyclic alkene groups and heterocyclic alkylene groups comprising a heterocyclic element selected from the group consisting of nitrogen and sulfur;
wherein said medicament is in nanoparticle form having a largest linear dimension of 1-999 nanometers.

2. The medicament of claim 1 wherein said nanoparticle includes in-vivo bone-targeting functionality.

3. The medicament of claim 1 wherein said phenothiazine comprises promethazine having the following structure:

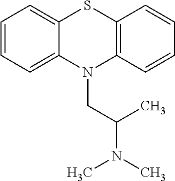

4. The medicament of claim 1 wherein said nanoparticles encapsulate said phenothiazines.

5. The medicament of claim 4 wherein said nanoparticles encapsulating said phenothiazine comprise a polymeric component which polymeric component has hydrophilic and/or hydrophobic type character.

6. The medicament of claim 1 wherein said phenothiazines are encapsulated in said nanoparticles by a polymer component comprising one of poly(lactic-co-glycolic) acid or poly(lactic-b-PEG).

7. The medicament of claim 6 wherein said poly(lactic-co-glycolic) acid has the following structure: 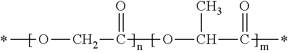
wherein the value of n and m is between 1-1000.

8. The medicament of claim 6 wherein said poly(lactic-b-PEG) has the following structure: 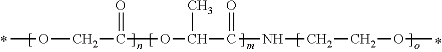
wherein the value of n, m or o is between 1-1000.

9. The medicament of claim 1 wherein said phenothiazines are encapsulated in said nanoparticles by a polymer component comprising a PLGA-alendroate polymer having the following structure: 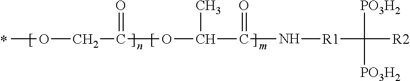
wherein the value of n or m is between 1-1000, R1 comprises a linking functionality providing covalent attachment of the indicated bisphosophonate functionality and R2 comprises an alkyl amino type group.

10. The medicament of claim 1 wherein said phenothiazine is a (+) enantiomer.

11. The medicament of claim 1 wherein said phenothiazines is a (−) enantiomer.

12. The medicament of claim 3 wherein said promethazine is a (+) enantiomer.

13. The medicament of claim 3 wherein said promethazine is a (−) enantiomer.

14. The medicament of claim 1 wherein said phenothiazine is combined in a pharmaceutically acceptable carrier.

15. A method of preventing or inhibiting a disease or condition comprising administering to a patient or animal having a risk of having a disease or condition associated with bone loss a therapeutically effective amount of a medicament comprising:
phenothiazines having the structure: 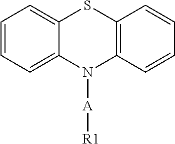
wherein A may be selected from the group consisting of linear or branched alkyls and/or linear or branched alkenyl groups having 1 to 5 carbon atoms;
R1 may be a tertiary amine or thiol group having a structure including N—(R2)₃ or S—(R2) wherein R2 comprises the same or different entities selected from the group consisting of hydrogen, alkyl groups, alkenyl groups having 1 to 4 carbon atoms, cyclic alkene groups and heterocyclic alkylene groups comprising a heterocyclic element selected from the group consisting of nitrogen and sulfur;
wherein said medicament is in nanoparticle form having a largest linear dimension of 1-999 nanometers and wherein said medicament is combined in a pharmaceutically acceptable carrier.

16. The method of claim 15 wherein said nanoparticle form includes bone targeting functionality.

17. The method of claim 15 wherein said phenothiazine comprises promethazine having the following structure:

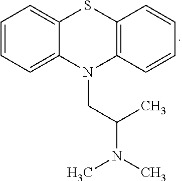

18. The method of claim 15 wherein said nanoparticles encapsulate said phenothiazines.

19. The method of claim 18 wherein said nanoparticles encapsulating said phenothiazine comprise a polymeric component which polymeric component has hydrophilic and/or hydrophobic type character.

20. The method of claim 15 wherein said phenothiazines are encapsulated in said nanoparticles by a polymer component comprising one of poly(lactic-co-glycolic) acid or poly(lactic-b-PEG).

21. The method of claim 20 wherein said poly(lactic-co-glycolic) acid has the following structure: 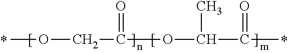
wherein the value of n and m is between 1-1000.

22. The method of claim 20 wherein said poly(lactic-b-PEG) has the following structure: 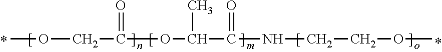
wherein the value of n, m or o is between 1-1000.

23. The method of claim 15 wherein said phenothiazines are encapsulated in said nanoparticles by a polymer component comprising a PLGA-alendroate polymer having the following structure: 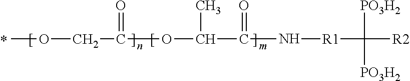
wherein the value of n or m is between 1-1000, R1 comprises a linking functionality providing covalent attachment of the indicated bisphosophonate functionality and R2 comprises an alkyl amino type group.

24. The method of claim 15 wherein said phenothiazine is a (+) enantiomer.

25. The method of claim 15 wherein said phenothiazines is a (−) enantiomer.

26. The method of claim 17 wherein said promethazine is a (+) enantiomer.

27. The method of claim 17 wherein said promethazine is a (−) enantiomer.

28. The method of claim 15 wherein said bone loss is monitored after treatment with said medicament by nuclear magnetic resonance to characterize bone porosity comprising:
placing a bone sample in an external magnetic field wherein said bone has a whole bone porosity comprising the porosity of the cortical, trabecular and marrow porosity for said bone;
providing an oscillating radio frequency electromagnetic field for exciting protons within said bone sample;
providing a receiver to receive signals in the form of data from the excited protons;
measuring the distribution of protons in said bone sample from said spectrum;
processing said data to characterize said whole bone porosity wherein said processing step includes determining the median T₂ relaxation times from said data.

29. A method for using nuclear magnetic resonance to characterize bone porosity comprising:
placing a bone sample in an external magnetic field wherein said bone has a whole bone porosity comprising the porosity of the cortical, trabecular and marrow porosity for said bone;
providing an oscillating radio frequency electromagnetic field for exciting protons within said bone sample;
providing a receiver to receive signals in the form of data from the excited protons;
measuring the distribution of protons in said bone sample from said spectrum;
processing said data to characterize said whole bone porosity wherein said processing step includes determining the median T₂ relaxation times from said data.

30. A method of preventing or inhibiting a disease or condition comprising administering to a patient or animal having a risk of having a disease or condition associated with bone loss a therapeutically effective amount of a nanoparticle medicament including in-vivo bone targeting functionality comprising:
phenothiazines having the structure: 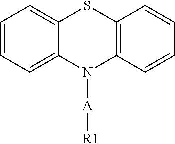
wherein A may be selected from the group consisting of linear or branched alkyls and/or linear or branched alkenyl groups having 1 to 5 carbon atoms;
R1 may be a tertiary amine or thiol group having a structure including N—(R2)₃ or S—(R2) wherein R2 comprises the same or different entities selected from the group consisting of hydrogen, alkyl groups, alkenyl groups having 1 to 4 carbon atoms, cyclic alkene groups and heterocyclic alkylene groups comprising a heterocyclic element selected from the group consisting of nitrogen and sulfur;
wherein said medicament is in nanoparticle form having a largest linear dimension of 1-999 nanometers and said nanoparticle form includes bone targeting functionality.
